(19) United States
(12) Patent Application Publication
(10) Pub. No.: US 2007/0031356 A1
(43) Pub. Date: Feb. 8, 2007

(54) Beta-carotene modulation of gene expression

(76) Inventors: Petra Buchwald Hunziker, Magden (CH); Regina Goralczyk, Grenzach-Wyhlen (DE); Willi Hunziker, Magden (CH); Martin Neeb, Weil am Rhein (DE); Georges Riss, Obermorschwiller (FR); Nicole Seifert, Rheinfelden (CH); Guido Steiner, Grenzach-Wyhlen (DE); Karin Wertz, Rheinfelden (DE)

Correspondence Address: Stephen M. Haracz, Esq.;BRYAN CAVE LLP, 33rd Floor, 1290 Avenue of the Americas, New York, NY, 10104, US

(21) Appl. No.: 11/479,232

(22) Filed: Jun. 30, 2006

Related U.S. Application Data
(60) Provisional application No. 60/696,225, filed on Jul. 1, 2005.

(51) Int. Cl. A61K 8/49; C12Q 1/68

(52) U.S. Cl. 424/59; 435/6

(57) ABSTRACT

Methods and compositions—particularly compositions containing β-carotene—for modulating the expression of genes that effect or influence cellular health or protect against cellular damage, e.g., skin aging, are provided. More particularly, a method is provided for screening for a compound that modulates an effect of UV irradiation on eukaryotic cells. Methods and compositions for ameliorating the effects of non-UV radiation induced skin aging and for modulating UVA-induced gene expression on skin aging are also provided. Further provided are methods and compositions for enhancing UVA-induced tanning of the skin, for promoting cell differentiation in UVA-irradiated cells of an organism, and for modulating stress-induced induction of a gene in an organism.

This application claims the benefit of U.S. Provisional Application 60/696,225, filed Jul. 1, 2005.

### FIELD OF THE INVENTION

The present invention relates to methods and compositions—particularly compositions containing β-carotene—for modulating the expression of genes that effect or influence, e.g., skin aging. The present invention also relates to methods for screening for compounds that modulate an effect of UV radiation on eukaryotic cells and/or promote cellular health.

### BACKGROUND OF THE INVENTION

UVA exposure is believed to cause skin aging mainly by singlet oxygen (¹O₂)-dependent pathways. ¹O₂ mediates gene regulation via the transcription factor AP-2 (Grether-Beck, 1996). Furthermore, like UVB/UVA2, UVA1 activates stress-activated protein kinases (Kick, 1996).

β-carotene has the potential to protect skin, first, because it is an excellent ¹O₂ quencher (Cantrell, 2003). During UVA exposure, skin is regularly exposed to ¹O₂, and is thus a most relevant tissue to test ¹O₂ quenching by β-carotene in living cells. Second, β-carotene scavenges reactive oxygen species (ROS) other than ¹O₂ (Krinsky, 2003). Third, β-carotene mildly reduces sun burn (Mathews-Roth, 1972; Stahl, 2000). In addition, β-carotene can be metabolized to retinoic acid (“RA”), a signaling molecule involved in skin maintenance.

β-carotene treatment produces a complex cellular response that includes the induction or inhibition of many genes. These genes are involved in various aspects of cellular and extracellular regulation. Many of these effects promote cellular health or protect against cellular damage.

Accordingly, it would be advantageous to provide a screening method that would allow for the identification of other compounds that produce similar effects on some or all of the genes that respond to treatment with β-carotene. In addition, it would be advantageous to provide methods and compositions to promote cellular health or protect against cellular damage.

### SUMMARY OF THE INVENTION

One embodiment of the present invention is a method for screening for a compound that modulates an effect of UV irradiation on eukaryotic cells. This method includes the steps of a) contacting a sample of eukaryotic cells with the compound to be evaluated, b) irradiating the cells from (a) with UV radiation, c) comparing a gene expression profile of the cells contacted with the compound to a gene expression profile of control cells that were not contacted with the compound prior to the irradiation step in (b), and d) correlating a difference in the gene expression profile of the cells exposed to the compound and the control cells that were not exposed to the compound with an ability of the compound to modulate an effect of UV irradiation on the cells.

Another embodiment of the present invention is a method for ameliorating the effects of non-UV radiation induced skin aging. This method includes administering to an organism in need thereof an amount of a compound selected from the group comprising or consisting of β-carotene, a precursor of β-carotene, a derivative of β-carotene, a salt of β-carotene, and combinations, which amount is effective to modulate a gene responsible for the non-UV radiation induced skin aging.

A further embodiment of the present invention is a composition for ameliorating the effects of non-UV radiation induced skin aging. This composition contains an amount of a compound selected from the group comprising or consisting of β-carotene, a precursor of β-carotene, a derivative of β-carotene, a salt of β-carotene, and combinations thereof, which amount is effective to modulate a gene responsible for the non-UV radiation induced skin aging.

An additional embodiment of the present invention is a method of modulating the effects of UVA-induced gene expression on skin aging. This method includes, prior to exposing the skin to UV-A radiation, administering to an organism an amount of a composition containing a compound selected from the group comprising or consisting of β-carotene, a precursor of β-carotene, a derivative of β-carotene, a salt of β-carotene, and combinations thereof, which amount is effective to modulate the effects of UVA-induced gene expression on skin aging.

Another embodiment of the present invention is a composition for modulating the effects of UVA-induced gene expression on skin aging. This composition includes an amount of a compound selected from the group comprising or consisting of β-carotene, a precursor of β-carotene, a derivative of β-carotene, a salt of β-carotene, and combinations thereof, which amount is effective to modulate the effects of UVA-induced gene expression on skin aging.

A further embodiment of the present invention is a method of enhancing UVA-induced tanning of the skin. This method includes administering to an organism, prior to exposure to UVA radiation, an amount of a composition containing a compound selected from the group comprising or consisting of β-carotene, a precursor of β-carotene, a derivative of β-carotene, a salt of β-carotene, and combinations thereof, which amount is effective to increase UVA-induced PAR-2 gene transcription.

An additional embodiment of the present invention is a composition for enhancing UVA-induced tanning. This composition contains an amount of a compound selected from the group comprising or consisting of β-carotene, a precursor of β-carotene, a derivative of β-carotene, a salt of β-carotene, and combinations thereof, which amount is effective to increase UVA-induced PAR-2 gene transcription.

Another embodiment of the present invention is a method for promoting cell differentiation in UVA-irradiated cells of an organism. This method includes administering to the organism in need thereof an amount of a compound selected from the group comprising or consisting of β-carotene, a precursor of β-carotene, a derivative of β-carotene, a salt of β-carotene, and combinations thereof, which amount is effective to downregulate transcription of a gene selected from the group comprising or consisting of BPAG1, integrin_{α6}, ILK, desmocollins, Cx45 and combinations thereof or to up regulate transcription of a gene selected from the group comprising or consisting of Cx31, KLF4, GADD153, and combinations thereof.

A further embodiment of the present invention is a composition for promoting cell differentiation in UVA irradiated cells of an organism. This composition contains an amount of a compound selected from the group comprising or consisting of β-carotene, a precursor of β-carotene, a derivative of β-carotene, a salt of β-carotene, and combinations thereof, which compound is effective to downregulate transcription of a gene selected from the group comprising or consisting of BPAG1, integrin_{α6}, ILK, desmocollins, Cx45, and combinations thereof or to up regulate transcription of a gene selected from the group comprising or consisting of Cx31, KLF4, GADD153, and combinations thereof.

An additional embodiment of the present invention is a method for modulating stress-induced induction of a gene in an organism. This method includes administering to the organism an amount of a compound selected from the group comprising or consisting of β-carotene, a precursor of β-carotene, a derivative of β-carotene, a salt of β-carotene, and combinations thereof, which amount is effective to modulate the stress-induced induction of the gene.

Another embodiment of the present invention is a composition for modulating stress-induced induction of a gene in an organism. This composition contains a compound selected from the group comprising or consisting of β-carotene, a precursor of β-carotene, a derivative of β-carotene, a salt of β-carotene, and combinations thereof, wherein the compound is present in the composition in an amount effective to modulate the stress-induced induction of the gene.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows β-carotene-induced inhibition of integrin_{α6} transcription in irradiated and unirradiated HaCaT cells (FIG. 1a) and enhancement of UVA-induced GADD34 (FIG. 1b) and GADD153 transcription (FIG. 1c). Cells were supplemented with β-carotene for 2 days prior to UVA irradiation (270 kJ/m²) either in normal PBS or D₂O-PBS. Gene expression 5 hours after irradiation was determined by quantitative real time polymerase chain reaction. (“QRT-PCR”). Values are geometric mean±standard error of three experiments.

FIG. 2 shows dose-dependent induction of caspase-3 activity in UVA-irradiated keratinocytes by β-carotene. Cells were supplemented with β-carotene for 2 days and prior to UVA irradiation (270 kJ/m²). Caspase-3 activity was determined at 5 hours after irradiation. Values are mean±standard error of an experiment with four replicates.

FIG. 3 shows a model of molecular events, as deduced from the microarray data below. FIG. 3a shows the effect of β-carotene treatment in unirradiated keratinocytes. FIG. 3b shows the effect of UVA-irradiation in keratinocytes. FIG. 3c shows the effect of β-carotene treatment in UVA-irradiated keratinocytes. Genes labeled red were upregulated and genes labeled green were downregulated by the treatment. β-carotene treatment quenched the effect of UVA irradiation for genes labeled blue.

FIG. 4 shows the relationship of the modes of action of 6-carotene to its influence on UVA-induced biological processes deduced from the experiments below.

### DETAILED DESCRIPTION OF THE INVENTION

One embodiment of the present invention is a method for screening for a compound that modulates an effect of UV irradiation on eukaryotic cells. This method includes the steps of a) contacting a sample of eukaryotic cells with the compound to be evaluated, b) irradiating the cells from (a) with UV radiation, c) comparing a gene expression profile of the cells contacted with the compound to a gene expression profile of control cells that were not contacted with the compound prior to the irradiation step in (b), and d) correlating a difference in the gene expression profile of the cells exposed to the compound and the control cells that were not exposed to the compound with an ability of the compound to modulate an effect of UV irradiation on the cells.

In the present invention, the genetic profile analyzed is a transcriptome profile. A complete transcriptome refers to the complete set of mRNA transcripts produced by the genome at any one time. Unlike the genome, the transcriptome is dynamic and varies considerably in differing circumstances due to different patterns of gene expression. Transcriptomics, the study of the transcriptome, is a comprehensive means of identifying gene expression patterns. The transcriptome analyzed can include the complete known set of genes transcribed, i.e. the mRNA content or corresponding cDNA of a host cell or host organism. The cDNA can be a chain of nucleotides, an isolated polynucleotide, nucleotide, nucleic acid molecule, or any fragment or complement thereof that originated recombinantly or synthetically and be double-stranded or single-stranded, coding and/or noncoding, an exon or an intron of a genomic DNA molecule, or combined with carbohydrate, lipids, protein or inorganic elements or substances. The nucleotide chain can be at least 5, 10, 15, 30, 40, 50, 60, 70, 80, 90 or 100 nucleotides in length. The transcriptome can also include only a portion of the known set of genetic transcripts. For example, the transcriptome can include less than 98%, 95, 90, 85, 80, 70, 60, or 50% of the known transcripts in a host. The transcriptome can also be targeted to a specific set of genes.

In the present invention, the screening process can include screening using an array or a microarray to identify a genetic profile. In the present invention, the transcriptome or gene expression profile can be analyzed by using known processes such as hybridization in blot assays such as northern blots. In the present invention, the process can include PCR-based processes such as RT-PCR that can quantify expression of a particular set of genes.

The process can include analyzing the transcriptome or gene expression profile using a microarray or equivalent technique. In this process, the microarray can include at least a portion of the transcribed genome of the host cell, and typically includes binding partners to samples from genes of at least 50% of the transcribed genes of the organism. More typically, the microarray or equivalent technique includes binding partners for samples from at least 80%, 90%, 95%, 98%, 99% or 100% of the transcribed genes in the genome of the host cell. However, it is also possible that the microarray can include binding partners only to a selected subset of genes from the genome, including but not limited to putative genes that control or influence cellular health or protect against cellular damage. A microarray or equivalent technique can typically also include binding partners to a set of genes that are used as controls, such as housekeeper genes. A microarray or equivalent technique can also include genes clustered into groups such as genes coding for immediate early genes, oxidative defense genes, extracellular matrix genes, pro-inflammatory genes, VEGF-related ligand and receptor genes, IFNα/β genes, interleukin genes, proteinase-activated receptor genes, prostaglandin synthesis and signalling genes, EGF-related ligand and receptor genes, FGF-related ligand and receptor genes, TGF-β-related ligand and receptor genes, Wnt signalling genes, IGF/insulin signalling genes, Jagged/Delta signalling genes, MAPK pathway genes, differentiation marker genes, cell cycle genes, apoptosis genes, and combinations thereof.

A microarray is generally formed by linking a large number of discrete binding partners, which can include polynucleotides, aptamers, chemicals, antibodies or other proteins or peptides, to a solid support such as a microchip, glass slide, or the like, in a defined pattern. By contacting the microarray with a sample obtained from a cell of interest and detecting binding of the binding partners expressed in the cell that hybridize to sequences on the chip, the pattern formed by the hybridizing polynucleotides allows the identification of genes or clusters of genes that are expressed in the cell. Furthermore, where each member linked to the solid support is known, the identity of the hybridizing partners from the nucleic acid sample can be identified. One strength of microarray technology is that it allows the identification of differential gene expression simply by comparing patterns of hybridization.

Examples of high throughput screening processes include hybridization of host cell mRNA or substantially corresponding cDNA, to a hybridizable array(s) or microarray(s). The array or microarray can be one or more array(s) of nucleic acid or nucleic acid analog oligomers or polymers. In the present invention, the array(s) or microarray(s) may be independently or collectively a host-cell-genome-wide array(s) or microarray(s), containing a population of nucleic acid or nucleic acid analog oligomers or polymers whose nucleotide sequences are hybridizable to representative portions of all genes known to encode or predicted as encoding genes that control or influence cellular health or protect against cellular damage in the host cell strain. A genome-wide microarray includes sequences that bind to a representative portion of all of the known or predicted open reading frame (ORF) sequences, such as from mRNA or corresponding cDNA of the host.

The oligonucleotide sequences or analogs in the array typically hybridize to the mRNA or corresponding cDNA sequences from the host cell and typically comprise a nucleotide sequence complimentary to at least a portion of a host mRNA or cDNA sequence, or a sequence homologous to the host mRNA or cDNA sequence. Single DNA strands with complementary sequences can pair with each other and form double-stranded molecules.

Microarrays generally apply the hybridization principle in a highly parallel format. Instead of one identified, thousands of different potential identifieds can be arrayed on a miniature solid support. Instead of a unique labeled DNA probe, a complex mixture of labeled DNA molecules is used, prepared from the RNA of a particular cell type or tissue. The abundances of individual labeled DNA molecules in this complex probe typically reflect the expression levels of the corresponding genes. In a simplified process, when hybridized to the array, abundant sequences will generate strong signals and rare sequences will generate weak signals. The strength of the signal can represent the level of gene expression in the original sample.

In the present invention, a genome-wide array or microarray may be used. The array may represent more than 50% of the open reading frames in the genome of the host, or more than 55%, 60%, 65%, 70%, 75%, 80%, 85%, 90%, 91%, 92%, 93%, 94%, 95%, 96%, 97%, 98%, 99% or 100% of the known open reading frames in the genome. The array may also represent at least a portion of at least 50% of the sequences known to encode protein in the host cell. Alternatively, the array represents more than 50% of the genes or putative genes of the host cell, or more than 55%, 60%, 65%, 70%, 75%, 80%, 85%, 90%, 91%, 92%, 93%, 94%, 95%, 96%, 97%, 98%, 99% or 100% of the known genes or putative genes. In the present invention, more than one oligonucleotide or analog can be used for each gene or putative gene sequence or open reading frame. In the present invention, these multiple oligonucleotide or analogs represent different portions of a known gene or putative gene sequence. For each gene or putative gene sequence, from about 1 to about 10000 or from 1 to about 100 or from 1 to about 50, 45, 40, 35, 30, 25, 20, 15, 10 or less oligonucleotides or analogs can be present on the array.

A microarray or a complete genome-wide array or microarray may be prepared according to any process known in the art, based on knowledge of the sequence(s) of the host cell genome, or the proposed coding sequences in the genome, or based on the knowledge of expressed mRNA sequences in the host cell or host organism.

For different types of host cells, the same type of microarray can be applied. The types of microarrays include complementary DNA (cDNA) microarrays (Schena, M. et al. (1995) Quantitative monitoring of gene expression patterns with a complementary DNA microarray. Science 270:467-70) and oligonucleotide microarrays (Lockhart, et al. (1996) Expression monitoring by hybridization to high-density oligonucleotide arrays. Nat Biotechnol 14:1675-80). For cDNA microarray, the DNA fragment of a partial or entire open reading frame is printed on the slides. The hybridization characteristics can be different throughout the slide because different portions of the molecules can be printed in different locations. For the oligonucleotide arrays, 20-80-mer oligos can be synthesized either in situ (on-chip) or by conventional synthesis followed by on-chip immobilization, however in general all probes are designed to be similar with regard to hybridization temperature and binding affinity (Butte, A. (2002) The use and analysis of microarray data. Nat Rev Drug Discov 1:951-60).

In analyzing the transcriptome profile or gene expression, the nucleic acid or nucleic acid analog oligomers or polymers can be RNA, DNA, or an analog of RNA or DNA. Such nucleic acid analogs are known in the art and include, e.g.: peptide nucleic acids (PNA); arabinose nucleic acids; altritol nucleic acids; bridged nucleic acids (BNA), e.g., 2′-O, 4′-C-ethylene bridged nucleic acids, and 2′-O, 4′-C-methylene bridged nucleic acids; cyclohexenyl nucleic acids; 2′,5′-linked nucleotide-based nucleic acids; morpholino nucleic acids (nucleobase-substituted morpholino units connected, e.g., by phosphorodiamidate linkages); backbone-substituted nucleic acid analogs, e.g., 2′-substituted nucleic acids, wherein at least one of the 2′ carbon atoms of an oligo- or poly-saccharide-type nucleic acid or analog is independently substituted with, e.g., any one of a halo, thio, amino, aliphatic, oxyaliphatic, thioaliphatic, or aminoaliphatic group (wherein aliphatic is typically C₁-C₁₀ aliphatic).

Oligonucleotides or oligonucleotide analogs in the array can be of uniform size and, for example, can be about 10 to about 1000 nucleotides, about 20 to about 1000, 20 to about 500, 20 to about 100, about 20, about 25, about 30, about 40, about 50, about 60, about 70, about 80, about 90 or about 100 nucleotides long.

The array of oligonucleotide probes can be a high density array comprising greater than about 100, or greater than about 1,000 or more different oligonucleotide probes. Such high density arrays can comprise a probe density of greater than about 60, more generally greater than about 100, most generally greater than about 600, often greater than about 1000, more often greater than about 5,000, most often greater than about 10,000, typically greater than about 40,000 more typically greater than about 100,000, and in certain instances is greater than about 400,000 different oligonucleotide probes per cm² (where different oligonucleotides refers to oligonucleotides having different sequences). The oligonucleotide probes range from about 5 to about 500, or about 5 to 50, or from about 5 to about 45 nucleotides, or from about 10 to about 40 nucleotides and most typically from about 15 to about 40 nucleotides in length. Particular arrays contain probes ranging from about 20 to about 25 oligonucleotides in length. The array may comprise more than 10, or more than 50, or more than 100, and typically more than 1000 oligonucleotide probes specific for each identified gene. In the present invention, the array may comprise at least 10 different oligonucleotide probes for each gene. Alternatively, the array may have 20 or fewer oligonucleotides complementary each gene. Although a planar array surface is typical, the array may be fabricated on a surface of virtually any shape or even on multiple surfaces.

The array may further comprise mismatch control probes. Where such mismatch controls are present, the quantifying step may comprise calculating the difference in hybridization signal intensity between each of the oligonucleotide probes and its corresponding mismatch control probe. The quantifying may further comprise calculating the average difference in hybridization signal intensity between each of the oligonucleotide probes and its corresponding mismatch control probe for each gene.

In some assay formats, the oligonucleotide probe can be tethered, i.e., by covalent attachment, to a solid support. Oligonucleotide arrays can be chemically synthesized by parallel immobilized polymer synthesis processes or by light directed polymer synthesis processes, for example on poly-L-lysine substrates such as slides. Chemically synthesized arrays are advantageous in that probe preparation does not require cloning, a nucleic acid amplification step, or enzymatic synthesis. The array includes test probes which are oligonucleotide probes each of which has a sequence that is complementary to a subsequence of one of the genes (or the mRNA or the corresponding antisense cRNA) whose expression is to be detected. In addition, the array can contain normalization controls, mismatch controls and expression level controls as described herein.

An array may be designed to include one hybridizing oligonucleotide per known gene in a genome. The oligonucleotides or equivalent binding partners can be 5′-amino modified to support covalent binding to epoxy-coated slides. The oligonucleotides can be designed to reduce cross-hybridization, for example by reducing sequence identity to less than 25% between oligonucleotides. Generally, melting temperature of oligonucleotides is analyzed before design of the array to ensure consistent GC content and Tₘ, and secondary structure of oligonucleotide binding partners is optimized. For transcriptome or gene expression profiling, secondary structure is typically minimized. An array may have each oligonucleotide printed at least two different locations on the slide to increase accuracy. Control oligonucleotides can also be designed based on sequences from different species than the host cell or organism to show background binding.

The samples in the genetic profile can be analyzed individually or grouped into clusters. The clusters can typically be grouped by similarity in gene expression. In the present invention, the clusters may be grouped individually as genes that are regulated to a similar extent in a host cell. The clusters may also include groups of genes that are regulated to a similar extent in a recombinant host cell, for example genes, that are up-regulated or down-regulated to a similar extent compared to a host cell or a modified or an unmodified cell. The clusters can also include groups related by gene or protein structure, function or, in the case of a transcriptome or gene expression array, by placement or grouping of binding partners to genes in the genome of the host.

Groups of binding partners or groups of genes or proteins analyzed can include, but are not limited to: immediate early genes, oxidative defense genes, extracellular matrix genes, pro-inflammatory genes, VEGF-related ligand and receptor genes, IFNα/β genes, interleukin genes, proteinase-activated receptor genes, prostaglandin synthesis and signalling genes, EGF-related ligand and receptor genes, FGF-related ligand and receptor genes, TGF-β-related ligand and receptor genes, Wnt signalling genes, IGF/insulin signalling genes, Jagged/Delta signalling genes, MAPK pathway genes, Differentiation marker genes, cell cycle genes, apoptosis genes, and combinations thereof. Genes in these groups include, but are not limited to: genes coding for putative or known C-FOS, FRA-1, JUN-D, JUN-B, MAF-F, C-MYC, OSR-1, GEM, DKK-1, GADD34, GADD153, IEX-1, TSSC3/IPL, TDAG51, MMP-1, MMP-3, MMP-10, serpinB1, lekti, PAR-2, VEGF, IL-6, HB-EGF, SMADs, EGFR HER3, Wnt5A, FGFR2, cyclin E, ODC, ID1-3, ID-4, RB, K167, thymidylate synthase, DNA ligase III, CENP-E, centromere and spindle protein genes, COL4, COL7, Cx31, BPAG1, integrin α6, KLF4, and ILK.

Another embodiment of the present invention is a method for ameliorating the effects of non-UV radiation induced skin aging. This method includes administering to an organism in need thereof an amount of a compound selected from the group comprising or consisting of β-carotene, a precursor of β-carotene, a derivative of β-carotene, a salt of β-carotene, and combinations thereof, which amount is effective to modulate a gene responsible for the non-UV radiation induced skin aging.

As used herein, the term “organism in need thereof” means an organism suffering from or susceptible to skin aging, for example, non-light induced skin aging. Preferably, the organism is a mammal, more preferably, a human.

As used herein, the terms “effective amount” “amount . . . effective” or like terms mean the amount of a composition or substance sufficient to produce modulation of the expression of the gene or genes of interest in the organism to which the composition or substance is administered. Preferably, an effective amount of β-carotene or other compound according to the present invention is from about 1 milligram to about 30 milligrams per day. More preferably, an effective amount of β-carotene is from about 5 milligrams to about 20 milligrams, even more preferably from about 10 milligrams to about 15 milligrams per day. In the present invention, “modulation,” “modulate,” or like terms mean an up regulation, down regulation or quenching of gene expression caused by α-carotene or other compound/composition of interest.

Non-limiting examples of genes responsible for non-UV radiation skin aging are genes selected from the group comprising or consisting of a member of the stress signal family of genes, a member of the ECM degradation family of genes, a member of the immune modulation family of genes, a member of the inflammation-causing family of genes, a member of the cellular differentiation family of genes, and combinations thereof. Preferably, the cellular differentiation family of genes is selected from the group comprising or consisting of growth factor signalling genes, cell cycle regulation genes, differentiation genes, apoptosis genes, and combinations thereof. Preferably, the growth factor signalling genes are selected from the group comprising or consisting of EGFR, HER-3, FGF3, FRZ-6, NOTCH3, BMP2a, Wnt5a, and combinations thereof and the cell cycle regulation genes are selected from the group comprising or consisting of G1, RB, p21, ID-2, DNA ligase III, DNA-PK G2/M, BUB1, and combinations thereof.

Preferably, the immune modulation and inflammation family of genes are selected from the group comprising or consisting of VEGF, IL-18, COX-2, and combinations thereof. Preferably, the ECM degradation family of genes is selected from the group comprising or consisting of MMP-1, MMP-10, and combinations thereof. Preferably, the stress signal family of genes is selected from the group comprising or consisting of JUN-B, FRA-2, NRF-2, GEM, EGRα, TSSC3/IPL, and combinations thereof.

A further embodiment of the present invention is a composition for ameliorating the effects of non-UV radiation induced skin aging. This compound contains an amount of a compound selected from the group consisting of β-carotene, a precursor of β-carotene, a derivative of β-carotene, a salt of β-carotene, and combinations thereof, which amount is effective to modulate a gene responsible for the non-UV radiation induced skin aging. In the present invention, other forms of β-carotene are also contemplated.

Effective dosage forms, modes of administration, and dosage amounts of compounds or compositions according to the present invention may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of animal, and like factors well known in the arts of medicine and veterinary medicine. In general, a suitable dose of a compound or composition according to the invention will be that amount of the compound or composition, which is the lowest dose effective to produce the desired effect. For example, an effective dose of β-carotene maybe administered as a single dose or as two, three, four, five, six or more sub-doses, administered separately at appropriate intervals throughout the day.

The compound or compositions of the present invention may be administered in any desired and effective manner: as pharmaceutical compositions for oral ingestion, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Preferably, the β-carotene is administered orally or topically. Further, the β-carotene may be administered in conjunction with other treatments. The β-carotene maybe encapsulated or otherwise protected against gastric or other secretions, if desired.

While it is possible for, e.g., the β-carotene of the invention to be administered alone, it is preferable to administer the β-carotene as a pharmaceutical formulation (composition). The pharmaceutically acceptable compositions of the invention comprise, e.g., β-carotene as an active ingredient in admixture with one or more pharmaceutically-acceptable carriers and, optionally, one or more other compounds, drugs, ingredients, and/or materials. Regardless of the route of administration selected, the compounds of the present invention are formulated into pharmaceutically-acceptable dosage forms by conventional methods known to those of skill in the art. See, e.g., Remington's Pharmaceutical Sciences (Mack Publishing Co., Easton, Pa.).

Pharmaceutical carriers are well known in the art (see, e.g., Remington's Pharmaceutical Sciences (Mack Publishing Co., Easton, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen β-carotene dosage form and method of administration can be determined using ordinary skill in the art.

The pharmaceutically acceptable compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) emulsifying and suspending agents; (21), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (22) propellants, such as chlorofluorohydrocarbons and volatile unsubstituted hydrocarbons, such as butane and propane; (23) antioxidants; (24) agents which render the formulation isotonic with the blood of the intended recipient, such as sugars and sodium chloride; (25) thickening agents; (26) coating materials, such as lecithin; and (27) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen β-carotene dosage form and method of administration may be determined using ordinary skill in the art.

Pharmaceutical formulations suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise, e.g., β-carotene in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug, it is desirable to slow its absorption from subcutaneous or intramuscular injection. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In the present invention, the compounds, e.g., β-carotene may be incorporated into various finished products, such as for example, a food, fortified food, functional food, food additive, clinical nutrition formulation, feed, fortified feed, functional feed, feed additive, beverage, dietary supplement, pharmaceutical, personal care product, nutraceutical, lotion, cream, spray, etc.

An additional embodiment of the present invention is a method of modulating the effects of UVA-induced gene expression on skin aging. This method includes, prior to exposure to UV-A radiation, administering to an organism an amount of a composition containing a compound selected from the group comprising or consisting of β-carotene, a precursor of β-carotene, a derivative of β-carotene, a salt of β-carotene, and combinations thereof, which amount is effective to modulate the effects of UV-A-induced gene expression on skin aging.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

Another embodiment of the present invention is a composition for modulating the effects of UVA-induced gene expression on skin aging. This composition includes an amount of a compound selected from the group comprising or consisting of β-carotene, a precursor of β-carotene, a derivative of β-carotene, a salt of β-carotene, and combinations thereof, which amount is effective to modulate the effects of UVA-induced gene expression on skin aging.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

A further embodiment of the present invention is a method of enhancing UVA-induced tanning of the skin. This method includes administering to an organism, prior to exposure to UVA radiation, an amount of a composition containing a compound selected from the group comprising or consisting of β-carotene, a precursor of β-carotene, a derivative of β-carotene, a salt of β-carotene, and combinations thereof, which amount is effective to increase UVA-induced PAR-2 gene transcription.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

An additional embodiment of the present invention is a composition for enhancing UVA-induced tanning. This composition contains an amount of a compound selected from the group comprising or consisting of β-carotene, a precursor of β-carotene, a derivative of β-carotene, a salt of β-carotene, and combinations thereof, which amount is effective to increase UVA-induced PAR-2 gene transcription.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

Another embodiment of the present invention is a method for promoting cell differentiation in UVA-irradiated cells of an organism. This method includes administering to the organism in need thereof an amount of a compound selected from the group comprising or consisting of β-carotene, a precursor of β-carotene, a derivative of β-carotene, a salt of β-carotene, and combinations thereof, which amount is effective to downregulate transcription of a gene selected from the group comprising or consisting of BPAG1, integrin_{α6}, ILK, desmocollins, Cx45 and combinations thereof or upregulate transcription of a gene selected from the group comprising or consisting of Cx31, KLF4, GADD153, and combinations thereof.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

A further embodiment of the present invention is a composition for promoting cell differentiation in UVA irradiated cells of an organism. This composition contains an amount of a compound selected from the group comprising or consisting of β-carotene, a precursor of β-carotene, a derivative of β-carotene, a salt of β-carotene, and combinations thereof, which compound is effective to downregulate transcription of a gene selected from the group comprising or consisting of BPAG1, integrin_{α6}, ILK, desmocollins, Cx45, and combinations thereof or to up regulate transcription of a gene selected from the group comprising or consisting of Cx31, KLF4, GADD153, and combinations thereof.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

An additional embodiment of the present invention is a method for modulating stress-induced induction of a gene in an organism. This method includes administering to the organism an amount of a compound selected from the group comprising or consisting of β-carotene, a precursor of β-carotene, a derivative of β-carotene, a salt of β-carotene, and combinations thereof, which amount is effective to modulate the stress-induced induction of the gene.

In the present embodiment, the organisms, amounts of the compound(s), e.g., β-carotene, a precursor of β-carotene, a derivative of 8-carotene, a salt of β-carotene, or a combination thereof, delivery routes, and composition forms are as defined above.

Another embodiment of the present invention is a composition for modulating stress-induced induction of a gene in an organism. This composition contains a compound selected from the group comprising or consisting of β-carotene, a precursor of β-carotene, a derivative of β-carotene, a salt of β-carotene, and combinations thereof, wherein the compound is present in the composition in an amount effective to modulate the stress-induced induction of the gene.

In the present embodiment, the organisms, amounts of the compound(s), e.g., β-carotene, a precursor of β-carotene, a derivative of β-carotene, a salt of β-carotene, or a combination thereof, delivery routes, and composition forms are as defined above.

The following examples are provided to further illustrate the compositions and methods of the present invention. These examples are illustrative only and are not intended to limit the scope of the invention in any way.

### EXAMPLES

### Example 1

UVA exposure is thought to cause skin aging mainly by singlet oxygen (¹O₂)-dependent pathways. Using microarray hybridization the effect of pretreatment with the ¹O₂ quencher β-carotene (1.5 μM) on prevention of UVA-induced gene regulation in HaCaT human keratinocytes was explored.

β-Carotene and UVA Treatment of Keratinocytes

The cell culture experiments were carried out as described (Wertz, 2004). Briefly, a subclone of passage 65 HaCaT keratinocytes, selected for differentiation capacity, was used at passages 16 to 23 after subcloning. 2×10⁵ cells were seeded per 60 millimeter dish. Starting the following day, the cells were pretreated for 2 days with β-carotene at 1.5 μM, a typical concentration in human plasma after moderate dietary supplementation (Thurmann, 2002).

β-carotene-containing medium was prepared as follows. Fresh all-E-β-carotene (DSM Nutritional Products, Kaiseraugst, Switzerland) stock solution in THF (containing 0.025% BHT; Fluka Chemie AG, Switzerland) was diluted 1:2 with ethanol and added to cell culture medium to a final concentration of 1.5 μM β-carotene. The solvent concentration in the medium was 0.5% for all treatments. β-carotene-containing medium was prepared fresh for the daily medium changes.

On day 3 of the experiment, the cells were irradiated with a Hönle sun lamp Sol 500 (270 kJ/m²; Dr. Hönle, Germany).

Cellular uptake of β-carotene from the culture medium was confirmed by HPLC analysis. Cells contained 20.06±5.66 pmol β-carotene/10⁶ cells after incubation with medium containing 1.85±0.09 μM β-carotene. During the 24 hours of incubation, the β-carotene concentration dropped to approximately 50% (not shown), irrespective of the presence of cells. No β-carotene was detected in placebo controls.

Affymetrix GeneChip® Analysis

Five independent, factorially designed cell irradiation experiments were analyzed by microarray hybridization. For each experiment, one chip was hybridized per treatment condition. GeneChip® analysis was done as described in Siler, 2004, which is incorporated by reference as if recited in full herein. Gene regulation by β-carotene and/or UVA was calculated relative to placebo.

Gene regulation is reported as “change factors”, defined as “(treatment/control)−1” (in case of an increase), or “-(control/treatment)+1” (in case of a decrease), or zero (in case of no change). Changes in gene expression were included in further analysis only if the change factor was ≧0.5 or ≦−0.5, and if unpaired t-tests yielded p values ≦0.05. Upregulations by a change factor of ≧0.5 are labeled bold, downregulations by a change factor of ≦−0.5 are labeled bold italics. (Table 1.) To identify the pathways affected by the treatments functional information on the genes was retrieved from public literature databases.

It was determined that 1458 genes were significantly regulated by at least one of the treatments. β-carotene regulated 381 genes. UVA radiation influenced 568 genes. 1142 genes were regulated by co-treatment with UVA radiation and β-carotene. Of these, 610 were not regulated by treatments with only UVA radiation or β-carotene alone.

UVA irradiation produced downregulation of growth factor signalling, moderate induction of proinflammatory genes, upregulation of immediate early genes including apoptotic regulators, and suppression of cell cycle genes. Of the 568 UVA-regulated genes, β-carotene reduced the UVA-induced effect for 143 genes, enhanced it for 180 genes, and had no effect for 245 genes. The different interaction modes imply that β-carotene/UVA interaction involved multiple mechanisms.

In unirradiated keratinocytes, gene regulations suggest that β-carotene reduced stress signals and extracellular matrix (“ECM”) degradation, and promoted keratinocyte differentiation. In irradiated cells, expression profiles indicate that β-carotene inhibited UVA-induced ECM-degradation, and enhanced UVA induction of tanning-associated PAR-2. Combination of β-carotene-promoted keratinocyte differentiation with the cellular “UV response” caused synergistic induction of cell cycle arrest and apoptosis.

β-carotene at physiological concentrations interacted with UVA radiation effects in keratinocytes by mechanisms that included, but were not restricted to ¹O₂ quenching. The retinoid effect of β-carotene was minor, indicating that the β-carotene effects reported here were predominantly mediated through vitamin A-independent pathways.

TABLE 1
Transcriptional response to β-carotene and/or UVA treatment.

| | | | | |
|---|---|---|---|---|
| | | | UVA | UVA and |
| Acc. No. | Gene | β-Carotene | Radiation | β-Carotene |

Immediate Early Genes/Oxidative Defense

| | | | | |
|---|---|---|---|---|
| AB020315 | DKK-1; dickkopf-1 | −0.3 | 0.76 | 1.4 |
| U10550 | GEM | [symbol] | 0.91 | 3.91 |
| AB017642 | OSR1; oxidative-stress responsive 1 | −0.18 | 0.74 | 0.59 |
| U60207 | KRS-2; stress responsive serine/threonine | [symbol] | [symbol] | −0.35 |
| | protein kinase | | | |
| AL022312 | ATF4; activating transcription factor 4 | 0.11 | 0.76 | 0.96 |
| V01512 | C-FOS | −0.14 | 0.81 | 2.49 |
| V01512 | C-FOS | −0.16 | 0.36 | 1.36 |
| X16707 | FRA-1 | 0.14 | 0.61 | 0.71 |
| X16706 | FRA-2 | [symbol] | 0 | −0.13 |
| J04111 | C-JUN | −0.06 | −0.13 | 0.93 |
| M29039 | JUNB | −0.38 | −0.19 | [symbol] |
| X51345 | JUNB | [symbol] | −0.28 | [symbol] |
| X56681 | JUND | 0.6 | 1.65 | 3.2 |
| X56681 | JUND | 0.01 | 1.31 | 1.83 |
| X56681 | JUND | 0 | 1.17 | 1.66 |
| AL021977 | MAF-F | −0.26 | 1.49 | 1.71 |
| V00568 | c-myc | 0.1 | 0.37 | 3.22 |
| V00568 | c-myc | 0.1 | 0.34 | 2.45 |
| M55914 | c-myc binding protein (mbp-1) | 0.5 | 0 | 0.54 |
| U40992 | hsp40; heat shock protein 40 | [symbol] | −0.36 | −0.07 |
| M32011 | NCF2; p67-phox; neutrophil oxidase factor | [symbol] | 0.26 | −0.16 |
| AF020761 | stimulator of Fe transport | 0.03 | 0.71 | 1.03 |
| M13699 | ceruloplasmin (ferroxidase) | 1 | 0.58 | 1 |
| X01060 | transferrin receptor | −0.05 | 0.49 | 0.91 |
| L20941 | ferritin heavy chain | 0.36 | −0.03 | 0.59 |
| U60319 | haemochromatosis protein (hla-h) | 0.15 | [symbol] | [symbol] |
| Y00451 | 5-aminolevulinate synthase | 0.08 | 0.53 | 0.41 |
| D38537 | protoporphyrinogen oxidase | −0.32 | −0.34 | [symbol] |
| J03824 | uroporphyrinogen III synthase | −0.15 | −0.16 | [symbol] |
| M57951 | bilirubin udp-glucuronosyltransferase | −0.25 | −0.15 | [symbol] |
| | isozyme 2 | | | |
| D16611 | coproporphyrinogen oxidase | −0.21 | −0.06 | [symbol] |
| L24123 | NRF1 | −0.27 | −0.08 | [symbol] |
| U13045 | NRF2, subunit beta 1 | [symbol] | −0.42 | −0.11 |
| X91247 | thioredoxin reductase | −0.08 | 0.53 | 1.48 |
| S62138 | GADD153 | −0.45 | 4.11 | 7.64 |
| Z50194 | TDAG51; PQ-rich protein; PHLDA1 | 0.27 | 2.3 | 6.19 |
| U83981 | GADD34 | −0.11 | 1.16 | 2.62 |
| AF001294 | TSSC3/IPL | [symbol] | 1.28 | 1.02 |
| AF035444 | TSSC3 | −0.44 | 0.51 | 0.7 |
| S81914 | IEX-1 | −0.08 | 1.65 | 0.91 |
| X78992 | ERF-2 | 0.31 | 0.54 | 0.99 |
| AF050110 | TIEG, EGRα | [symbol] | 0.3 | −0.11 |

Extracellular Matrix

| | | | | |
|---|---|---|---|---|
| X07820 | MMP10 | [symbol] | 3.59 | 2.2 |
| X05232 | MMP-3 | −0.46 | 1.23 | 0.93 |
| M13509 | MMP-1 | [symbol] | 0.06 | −0.22 |
| M93056 | serpinB1 | 0.7 | 0.1 | 0.57 |
| AJ228139 | Lekti | 0.48 | 0.27 | 0.75 |

Inflammation

| | | | | |
|---|---|---|---|---|
| U88879 | TLR3; toll-like receptor 3 | 0.6 | [symbol] | [symbol] |

VEGF-Related Ligands and Receptors

| | | | | |
|---|---|---|---|---|
| AF024710 | VEGF | 0.02 | 2.35 | 1.86 |
| AF022375 | VEGF | [symbol] | 1.1 | 0.78 |
| M63978 | VEGF | −0.48 | 0.9 | 1.09 |
| AF035121 | VEGFR2; VEGF receptor 2; KDR; FLK-1kdr/flk-1 | 0.6 | −0.18 | −0.28 |
| M36711 | AP-2α | −0.02 | 0.04 | [symbol] |

IFNα/β

| | | | | |
|---|---|---|---|---|
| M14660 | IFIT2; ISG-54K; (interferon stimulated gene) | 0.18 | 2.09 | 1.17 |
| M14660 | IFIT2; ISG-54K; (interferon stimulated gene) | −0.21 | 0.84 | 0.58 |
| AF026941 | IFIT4; IFI60; cig5; RIGG | −0.23 | 15.2 | 5.46 |
| L05072 | IRF-1; interferon regulatory factor 1 | 0.16 | 0.77 | 0.29 |
| U53831 | IRF-7b; interferon regulatory factor 7b | 0.16 | 0.61 | 0.49 |
| AJ225089 | TRIP14; ′2-5′ oligoadenylate synthetase | −0.07 | 1.36 | 0.67 |
| M24594 | IFIT1; IFI56 | 0 | 0.19 | [symbol] |
| M24594 | IFIT1; IFI56 | 0.05 | 0.23 | [symbol] |
| M97935 | IRF-9; p48; ISGF3γ; Interferon-stimulated | −0.18 | −0.11 | [symbol] |
| | transcription factor 3γ | | | |

Interleukins

| | | | | |
|---|---|---|---|---|
| X04430 | IL6; Interleukin 6; IFNβα2a | 0.6 | 0.65 | 2.29 |
| D49950 | IL18; IGIF (IFNγ inducing factor) | [symbol] | 0.43 | −0.18 |
| X52560 | C/EBPβ; NF-IL6 | −0.28 | 0.96 | 0.83 |
| M83667 | C/EBPδ; NF-IL6-β | 0.04 | 0.55 | 0.31 |
| U20240 | C/EBPγ | −0.09 | 0.65 | 0.71 |
| S78771 | NF-κB subunit | −0.1 | 0.45 | 0.56 |
| X61498 | NF-κB subunit | −0.07 | 0.45 | 0.7 |
| S76638 | NFκB; p50 | −0.46 | 0.47 | 0.55 |

Proteinase-Activated Receptors

| | | | | |
|---|---|---|---|---|
| M62424 | PAR-1; thrombin receptor | [symbol] | 0.13 | −0.05 |
| D10923 | HM74; PAR1-related | 0.18 | [symbol] | [symbol] |
| AF055917 | PAR-4; protease-activated receptor 4 | −0.49 | −0.44 | [symbol] |
| U67058 | PAR-2; proteinase activated receptor-2 | 0.03 | 2.92 | 3.32 |
| U34038 | PAR-2; proteinase activated receptor-2 | −0.27 | 1.65 | 1.91 |
| U34038 | PAR-2; proteinase activated receptor-2 | −0.03 | 1.26 | 1.34 |

Prostaglandin Synthesis and Signalling

| | | | | |
|---|---|---|---|---|
| U04636 | COX-2, cyclooxygenase-2 | [symbol] | −0.18 | 0.2 |

EGF-Related Ligands and Receptors

| | | | | |
|---|---|---|---|---|
| M60278 | HB-EGF; heparin-binding egf-like growth factor | 0.33 | 1.53 | 3.32 |
| X00588 | EGFR; precursor of epidermal growth factor receptor | [symbol] | −0.34 | [symbol] |
| H06628 | ERBB3 precursor; similar to | 0 | −0.07 | [symbol] |
| M34309 | HER3; epidermal growth factor receptor (her3) | −0.28 | 0.04 | [symbol] |
| M34309 | HER3; epidermal growth factor receptor (her3) | [symbol] | −0.04 | [symbol] |

FGF-Related Ligands and Receptors

| | | | | |
|---|---|---|---|---|
| M27968 | bFGF; basic fibroblast growth factor; FGF2 | 0.1 | 0.91 | 0.82 |
| M87770 | FGFR2; FGF receptor 2 | 0.08 | [symbol] | −0.48 |
| M64347 | FGFR3; FGF receptor | [symbol] | −0.2 | [symbol] |

TGFβ-Related Ligands and Receptors

| | | | | |
|---|---|---|---|---|
| X02812 | TGFβ; transforming growth factor β | 0.9 | 0.32 | 0.46 |
| M22489 | BMP2a; bone morphogenetic protein 2a (bmp-2a) | [symbol] | 0.1 | 0.16 |
| M62302 | GDF-1; growth/differentiation factor 1 (gdf-1) | [symbol] | −0.14 | −0.43 |
| U59423 | SMAD1 | −0.27 | −0.34 | [symbol] |
| U68019 | SMAD3 | 0.5 | −0.29 | 0.01 |
| U68019 | SMAD3 | 0.18 | [symbol] | −0.09 |
| U44378 | SMAD4 | [symbol] | 0.06 | [symbol] |
| U59913 | SMAD5 | [symbol] | −0.3 | [symbol] |
| AF035528 | SMAD6 | −0.47 | [symbol] | [symbol] |
| AF010193 | SMAD7 | −0.19 | −0.27 | [symbol] |

WNT Signalling

| | | | | |
|---|---|---|---|---|
| I20861 | WNT5A | −0.47 | [symbol] | [symbol] |
| I20861 | WNT5A | [symbol] | [symbol] | [symbol] |
| I37882 | frizzled-2 | −0.03 | −0.27 | [symbol] |
| AB012911 | frizzled-6 | [symbol] | [symbol] | [symbol] |

IGF/Insulin Signalling

| | | | | |
|---|---|---|---|---|
| M35878 | IGF-BP 3; insulin-like growth factor-binding | 0.29 | 1.95 | 1.64 |
| | protein-3 gene | | | |
| M35878 | IGF-BP 3; insulin-like growth factor-binding | 0.22 | 1.81 | 1.73 |
| | protein-3 gene | | | |
| X96584 | NOV | [symbol] | 2.43 | 0.72 |

Jagged/Delta Signalling

| | | | | |
|---|---|---|---|---|
| AF029778 | jagged2 (jag2) | −0.02 | [symbol] | [symbol] |
| U97669 | NOTCH3 | [symbol] | −0.42 | [symbol] |

MAPK Pathway

| | | | | |
|---|---|---|---|---|
| M54968 | K-RAS | [symbol] | −0.23 | [symbol] |
| X02751 | N-RAS | [symbol] | 0.1 | −0.01 |
| D87116 | MAPKK3b; MKK3b | −0.06 | 0.38 | 0.62 |
| L35263 | MAPK14; p38; csaids binding protein (csbp1) | −0.38 | −0.25 | [symbol] |
| U09759 | MAPK9; JNK2 | −0.36 | −0.08 | [symbol] |
| U71087 | MAPKK MEK5b | −0.16 | −0.42 | [symbol] |
| D45906 | LIM kinase 2 (limk-2) | −0.3 | −0.02 | [symbol] |
| U43195 | p160ROCK | −0.28 | −0.31 | [symbol] |
| U67156 | MAPKKK5; ASK1 | −0.06 | [symbol] | [symbol] |
| U48807 | MAP kinase phosphatase (mkp-2) | 0 | 1.1 | 1.12 |
| U15932 | DUSP5 | −0.15 | 1.87 | 2.84 |
| X93921 | DUSP7 | −0.46 | 1.13 | 0.99 |

Differentiation Markers

| | | | | |
|---|---|---|---|---|
| AF019084 | keratin 2e (KRT2E); Keratin 2A | −0.46 | −0.17 | [symbol] |
| M21389 | keratin 5 | 0.9 | 0.11 | 0.81 |
| J00124 | keratin 15 | 1 | −0.11 | 0.96 |
| M28439 | keratin 16 | [symbol] | 0.08 | −0.38 |
| Z19574 | keratin 17 | 0.04 | 0.03 | 0.6 |
| M69225 | BPAG1; bullous pemphigoid antigen | [symbol] | 0.91 | 0.12 |
| M91669 | bullous pemphigoid autoantigen bp180 | −0.45 | 0.07 | [symbol] |
| X56807 | DSC2; desmocollin type 2a and 2b | [symbol] | 0.35 | −0.29 |
| D17427 | desmocollin type 4 | [symbol] | −0.19 | [symbol] |
| X53586 | integrin α6 | [symbol] | 0 | [symbol] |
| S66213 | integrin α6b | [symbol] | 0.04 | −0.46 |
| S66213 | integrin α6b | [symbol] | −0.16 | [symbol] |
| U40282 | ILK; integrin-linked kinase | −0.23 | −0.13 | [symbol] |
| AF099730 | connexin 31 | 0.17 | 1.91 | 2.3 |
| U03493 | connexin 45 | −0.06 | 0.68 | 0.26 |
| X05610 | collagen type IV, α-2 (COL4A2) | −0.13 | [symbol] | [symbol] |
| M58526 | collagen type IV, α-5 (COL4A5) | [symbol] | [symbol] | [symbol] |
| D21337 | collagen type IV, α-6 (COL4A6) | −0.44 | −0.18 | [symbol] |
| L02870 | collagen type VII, α-1 (COL7A1) | −0.21 | −0.15 | [symbol] |
| U70663 | KLF4; EZF (epithelial Zn finger) | −0.17 | 1.99 | 3.46 |

Cell Cycle
G1 Phase

| | | | | |
|---|---|---|---|---|
| M73812 | cyclin E | −0.23 | 1.6 | 1.75 |
| AF091433 | cyclin E2 | −0.25 | 0.89 | 0.26 |
| M33764 | ornithine decarboxylase | −0.2 | 1.25 | 0.57 |
| X16277 | ornithine decarboxylase | −0.32 | 0.73 | 0.37 |
| X77743 | CDK activating kinase | −0.01 | 0.4 | 0.57 |
| U22398 | CDK-inhibitor p57KIP2 (KIP2) mrna | −0.22 | 1.25 | 0.79 |
| U03106 | p21; wild-type p53 activated fragment-1 (WAF1) | [symbol] | 0.26 | 0.21 |
| L25876 | CIP2; CDKN3 | −0.18 | −0.47 | [symbol] |
| X55504 | NOL1; p120 nucleolar antigen | 0.04 | 0.52 | 0.94 |
| AB024401 | p33; ING1b | −0.47 | 0.66 | 0.64 |
| L49229 | RB1 | [symbol] | [symbol] | [symbol] |
| X74594 | RB2/p130 | [symbol] | [symbol] | [symbol] |
| AL021154 | ID3; HEIR1 | −0.43 | [symbol] | [symbol] |
| X77956 | ID1 | −0.01 | [symbol] | [symbol] |
| X77956 | ID1 | −0.11 | [symbol] | [symbol] |
| D13891 | ID-2H | [symbol] | [symbol] | [symbol] |
| AL022726 | ID-4 | −0.08 | [symbol] | −0.49 |

S Phase: DNA Integrity Checkpoint, DNA Replication and Repair

| | | | | |
|---|---|---|---|---|
| L20046 | ERCC5; excision repair protein | −0.48 | −0.42 | [symbol] |
| U47077 | DNA-PK, catalytic subunit | [symbol] | −0.32 | [symbol] |
| M30938 | KU (p70/p80) | −0.18 | −0.28 | [symbol] |
| U40622 | XRCC4 | −0.05 | [symbol] | −0.01 |
| X65550 | mKI67a mrna (long type) for antigen of | −0.3 | [symbol] | [symbol] |
| | monoclonal antibody KI-67 | | | |
| X65550 | mKI67a mrna (long type) for antigen of | −0.17 | [symbol] | [symbol] |
| | monoclonal antibody KI-67 | | | |
| X67098 | rTS α | 0.13 | [symbol] | −0.47 |
| X02308 | thymidylate synthase | −0.12 | −0.23 | [symbol] |
| X84740 | DNA ligase III | [symbol] | −0.19 | [symbol] |
| X06745 | DNA polymerase α-subunit | −0.43 | −0.36 | [symbol] |
| X74331 | DNA primase (subunit p58) | −0.16 | [symbol] | −0.41 |
| L07493 | RPA; replication protein A 14kda subunit (rpa) | −0.04 | −0.08 | [symbol] |
| L47276 | α topoisomerase truncated-form | −0.43 | [symbol] | [symbol] |
| J04088 | TOP2; topoisomerase II | −0.22 | [symbol] | [symbol] |

G2/M Phase

| | | | | |
|---|---|---|---|---|
| U14518 | CENP-A; centromere protein-A | −0.13 | [symbol] | [symbol] |
| Z15005 | CENP-E; centromere protein-E | −0.37 | [symbol] | [symbol] |
| U30872 | CENP-F; mitosin | −0.42 | [symbol] | [symbol] |
| AF083322 | CEP110; centriole associated protein | 0.15 | [symbol] | [symbol] |
| AF011468 | STK15; BTAK | −0.18 | [symbol] | [symbol] |
| X62048 | WEE1 | [symbol] | 0.38 | 0.05 |
| AF053305 | BUB1; mitotic checkpoint kinase | [symbol] | [symbol] | [symbol] |
| AF053306 | MAD3L; mitotic checkpoint kinase | −0.18 | [symbol] | [symbol] |
| U37426 | KINESIN_LIKE 1; KNSL1; HKSP; EG5 | −0.04 | [symbol] | [symbol] |
| D14678 | KINESIN-LIKE 2; HSET | −0.04 | −0.3 | [symbol] |
| D14678 | KINESIN-LIKE 2; HSET | 0.03 | −0.42 | [symbol] |
| AL021366 | KINESIN-LIKE 2; HSET | −0.34 | [symbol] | [symbol] |
| X67155 | KINESIN-LIKE 5; KNSL5; MKLP-1; mitotic | −0.15 | [symbol] | [symbol] |
| | kinesin-like protein-1 | | | |
| U63743 | KINESIN-LIKE 6; KNSL6; MCAK; mitotic | −0.15 | [symbol] | [symbol] |
| | centromere-associated kinesin | | | |

Apoptosis

| | | | | |
|---|---|---|---|---|
| U19599 | BAXδ | 0.6 | −0.23 | 0.67 |
| L22475 | BAXγ | 0.8 | 0.17 | −0.34 |
| AB020735 | ENDOGL-2 | 0.8 | 0.35 | 0.41 |
| D90070 | NOXA | −0.26 | 0.85 | 0.74 |
| U67319 | caspase 7 | −0.09 | 0.62 | 0.31 |
| M96954 | TIAR; nucleolysin tiar | −0.45 | [symbol] | −0.08 |
| U13022 | caspase 2, ICH-1S | −0.31 | −0.23 | [symbol] |
| AF001433 | Requiem | 0.5 | 0.15 | 0.32 |
| U83857 | AAC11 | [symbol] | −0.19 | −0.05 |
| U37518 | TRAIL; TNF-related apoptosis inducing ligand | 0.15 | [symbol] | [symbol] |
| U77845 | TRIP | −0.06 | 0.04 | [symbol] |
| U84388 | CRADD; death domain containing protein | −0.25 | −0.46 | [symbol] |
| L41690 | TRADD; TNF receptor-1 associated protein | 0.23 | −0.19 | [symbol] |
| U79115 | RAIDD; death adaptor molecule | −0.22 | −0.4 | [symbol] |
| AF005775 | CLARP; CFLAR, alternatively spliced | −0.09 | −0.44 | [symbol] |

RA Targets

| | | | | |
|---|---|---|---|---|
| AF061741 | RETSDR1; retinal short-chain dehydrogenase/reductase | 1.1 | [symbol] | −0.06 |
| AJj005814 | HOXA7 | −0.43 | −0.31 | [symbol] |
| S82986 | HOXC6 | −0.07 | −0.11 | [symbol] |
| X59373 | HOXD4 | [symbol] | [symbol] | [symbol] |
| AF017418 | MEIS2 | 0.26 | [symbol] | [symbol] |
| M64497 | COUP-TF II; ARP1; apoA1 regulatory protein | 0.33 | 0.27 | 0.81 |
| U37146 | SMRT | −0.33 | [symbol] | [symbol] |
| X52773 | RXRα | −0.2 | [symbol] | [symbol] |
| U66306 | RXRα | −0.2 | −0.43 | [symbol] |

A) β-Carotene Effects In Unirradiated Keratinocytes:
β-Carotene Reduced Stress Responses

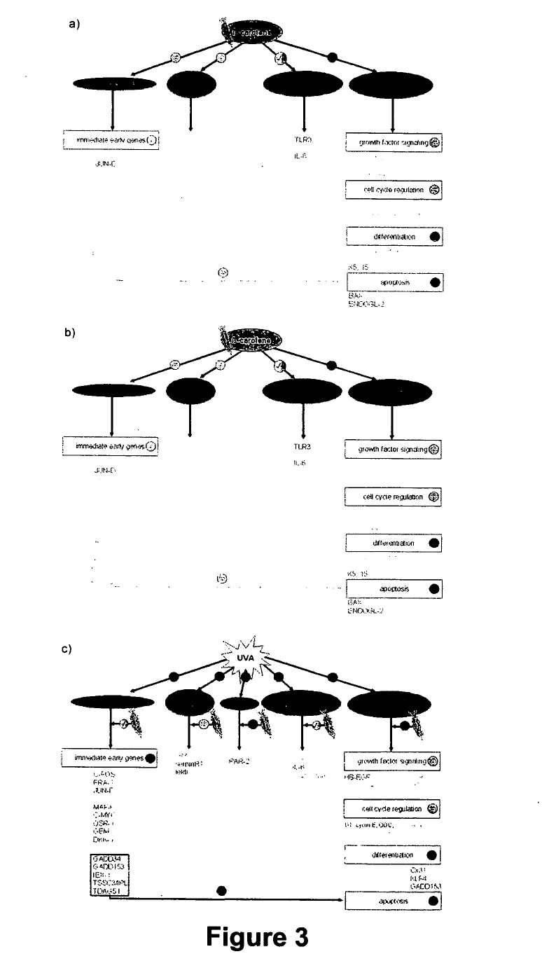

Stress stimuli, like UV irradiation or oxidative stress, e.g., resulting from ROS production in the respiratory chain, elicit a cellular stress response, leading to the induction of immediate early genes. β-carotene downregulated several immediate early genes (GEM, KRS-2, JUN-B, FRA-2, EGRα) and oxidative stress defense genes (NCF2, NRF2β1). This suggests that β-carotene reduced cellular stress including oxidative stress in unirradiated keratinocytes. (FIG. 3a.)

β-Carotene Reduced Basal MMP-10 Expression

Degradation of ECM molecules by matrix metalloproteases (MMPs) in skin is a key process in skin aging. β-carotene reduced the basal expression of MMP-10. This was confirmed by QRT-PCR in independent experiments (Wertz, 2004). MMP-10 cleaves various ECM molecules, but also activates other MMPs. Due to its broad substrate specificity, MMP-10 is likely involved in MMP-mediated skin aging.

Together with the finding that β-carotene mildly reduces basal MMP-1 expression (Wertz, 2004), this indicated that β-carotene reduces ECM degradation in unirradiated skin, and can therefore delay skin aging.

β-Carotene Promoted Normal Keratinocyte Differentiation

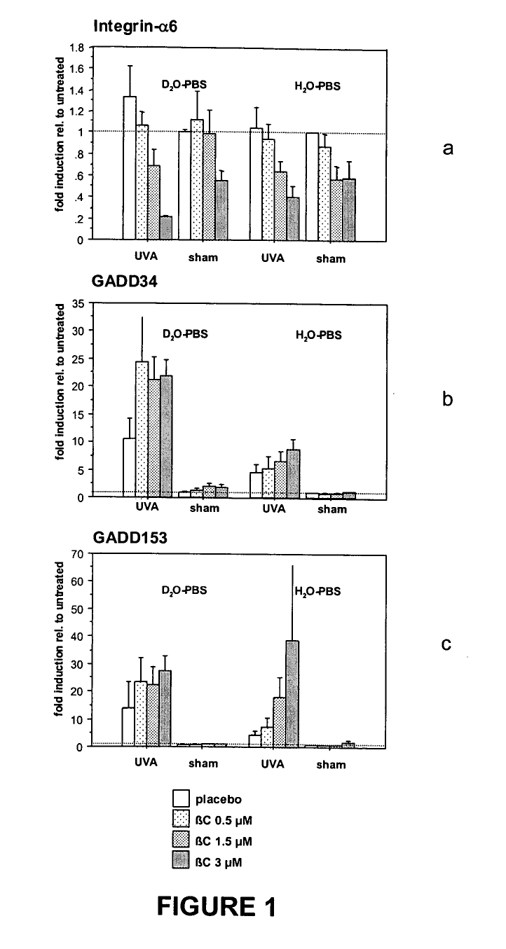

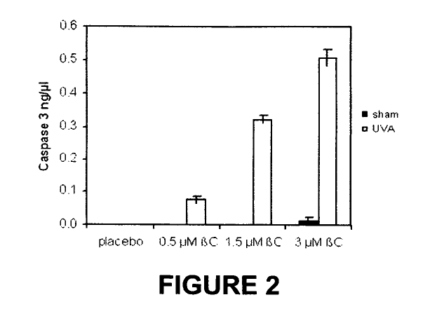

The response of HaCaT cells to β-carotene treatment was consistent with the cells undergoing differentiation. First, β-carotene downregulated genes associated with growth factor signaling (e.g., EGFR, NOTCH3, BMP2a, and Wnt5a) and cell cycle regulation (e.g., ID-2, DNA ligase III, and BUB1). Second, β-carotene regulated marker genes for physiological keratinocyte differentiation. Keratin 15 transcription was decreased and transcription of basement membrane collagen COL4A5 and the hemidesmosomal cell adhesion molecules BPAG1 and integrin α6 was decreased. QRT-PCR confirmed downregulation of integrin_{α6} (FIG. 1a). Since keratinocyte differentiation involves apoptosis, it is interesting that β-carotene upregulated several proapoptotic genes (Bax, endogl-2, requiem). This was counterbalanced, in part, by downregulation of immediate early genes, some of which favor apoptosis (e.g., TSSC3/IPL, EGRα). Apparently, β-carotene treatment prepared cells for apoptosis, but was not sufficient to induce apoptosis, as confirmed in a functional apoptosis assay (FIG. 2; unirradiated cells). This indicated that β-carotene promoted differentiation, but did not induce terminal differentiation in keratinocytes. β-Carotene Differentially Regulated Immune Modulators

β-carotene reportedly stimulates immune function (Hughes, 2001). β-carotene upregulated TLR3, a receptor involved in innate immunity, and IL-6, an important regulator of inflammation, keratinocyte growth, and wound healing. β-carotene mildly downregulated VEGF, a key angiogenic factor, and COX-2, the rate-limiting enzyme in prostaglandin synthesis. Moreover, β-carotene downregulated IL-18, an IL-12-related growth and differentiation factor for Th1 cells. Overall, β-carotene differentially regulated inflammatory signals in unirradiated keratinocytes.

β-Carotene Acted Predominantly Via RA-Independent Pathways

Among presumed RA-regulated genes, only retinol short chain dehydrogenase 1 (retSDR1) was induced by β-carotene. Other known RA targets (Balmer, 2002) were either not altered by β-carotene, or were downregulated (e.g., HOXD4), indicating that the effects of β-carotene described here were mainly RA-independent.

B) β-Carotene Effects In UVA-Irradiated Keratinocytes

β-carotene Interacts with UVA by Multiple Mechanisms

UVA irradiation elicited downregulation of growth factor-dependent signalling cascades, moderate induction of proinflammatory genes, induction of immediate early genes including apoptotic regulators, and suppression of cell cycle genes (FIG. 3b). He et al. (2004) made very similar observations in UVA-irradiated HaCaT cells. Of the 568 UVA-regulated genes, β-carotene quenched the UVA effect on 143 genes, i.e. they had expression profiles expected for ¹O₂-induced genes. On the other hand, β-carotene enhanced the UVA effect for 180 genes and had no influence on UVA regulation of 245 genes. These different modes of interference imply several mechanisms of UVA/β-carotene interaction.

α-Carotene Inhibited Expression of MMP-10 and Promoted Expression of Protease Inhibitors

Chronic sun exposure causes degradation of ECM proteins by inducing MMPs in skin, leading to premature skin aging. In our experiments, UVA irradiation induced MMP-10. β-carotene inhibited MMP-10 expression in UVA-irradiated keratinocytes. MMP-10 induction involves ¹O₂, and β-carotene dose-dependently inhibited MMP-10 induction by UVA/D₂O. Hence, β-carotene acts as a ¹O₂ quencher in living cells. β-carotene also reduced the basal and ¹O₂-induced expression of MMP-1 and downregulated UVA induction of MMP-3 (Wertz, 2004). Furthermore, β-carotene upregulated the protease inhibitors Lekti and serpinB1. TIMP-1, a likely MMP-10 inhibitor, was not influenced by the treatments.

Overall, the data indicated that β-carotene diminished UVA-induced ECM degradation, indicating that β-carotene at physiological concentrations may delay photoaging. Green and coworkers provided preliminary clinical evidence that β-carotene supplementation may indeed reduce wrinkling. (D Battistutta, G M Williams and A C Green: Effectiveness of daily sunscreen application and β-carotene intake for prevention of photoaging: a community-based randomised trial. International Congress on Photobiology; 28th Annual American Society for Photobiology Meeting, 2000, San Francisco).

β-Carotene Differentially Regulated Proinflammatory Genes

The cellular UV response includes induction of proinflammatory cytokines, but also immune suppression. β-carotene prevents UV-induced immune suppression (Fuller, 1992) and alleviates erythema after sun exposure (Gollnick, 1996; Stahl, 2000).

UVA induced mild signs of inflammation. β-carotene reduced UVA upregulation of VEGF and IFNα/Iβ targets. VEGF induction by UVA relies on an AP-2 site in the VEGF promoter (Gille, 2000), suggesting a 102-dependent regulation. VEGF downregulation may explain how β-carotene reduces erythema formation after sun exposure. IL-6 expression was weakly upregulated by UVA and enhanced by β-carotene. IL-6 is induced by IL-1 via a ¹O₂-dependent positive autoregulatory loop (Wlaschek, 1994). IL-6 can also be induced by SAPK/JNK signaling (Kick, 1996). As β-carotene did not quench the UVA induction of JNK/SAPK target genes, it appears that increased IL-6 induction by UVA and β-carotene occurred through JNK/SAPK signaling instead of the ¹O₂-dependent loop. IL-6 induction is expected to counteract the β-carotene-mediated VEGF reduction, thus impeding a stronger protection against erythema by β-carotene.

β-carotene Enhanced UVA Induction of PAR-2

PAR-2, a receptor required for tanning, was expectedly induced by UVA and further increased by β-carotene. Tronnier et al. (1984) report that carotenodermia positively influences pigmentation disorders independent of tanning. Raab, et al. (1985) and Postaire, et al. (1997), however, found an increased melanin content in skin after supplementation with β-carotene-containing antioxidant mixtures. β-carotene enhanced UVA induction of PAR-2 explains how carotenoid supplementation increases tanning after sun exposure.

β-carotene Acted Predominantly Via RA-Independent Pathways

UVA depletes cellular retinol stores (Sorg, 2002), possibly leading to reduced RA availability. Accordingly, RA target genes (Balmer, 2002) were downregulated by UVA irradiation. Except for retSDR1, β-carotene did not restore expression of RA target genes. HaCaT cells produce low amounts of retinoid activity from β-carotene (Wertz, 2004), rendering HaCaT cells an excellent model to evaluate provitamin A-independent functions of β-carotene.

β-Carotene Further Promoted Differentiation in Irradiated Keratinocytes

Expression of differentiation markers indicated that β-carotene promoted keratinocyte differentiation more strongly in UVA-irradiated cells than in unirradiated cells. UVA/β-carotene treatment downregulated more genes encoding basement membrane collagens than did the single treatments. Downregulation of BPAG1, integrin_{α6}, ILK, desmocollins, and Cx45, as well as upregulation of Cx31, KLF4 and GADD153 also indicate keratinocyte differentiation. This effect may render combined β-carotene/UVA treatment a promising therapy for skin disorders associated with disturbed differentiation, e.g., psoriasis.

β-Carotene Did Not Prevent UVA-Induced Stress Signals

Activation of JNK/SAPK, NFκB, and induction of their target genes are hallmarks of the cellular UV response. Massive transcriptional counterregulation of these signaling pathways occurred upon UVA irradiation. Expression profiles of protein kinases and phosphatases, and upregulation of target genes (C-FOS, FRA-1, JUND, ATF4, MAF-F, DKK-1, GEM) are consistent with a stress response induced by SAPK/JNK activation. β-carotene did not inhibit these UVA effects and enhanced some.

Few genes associated with oxidative stress were regulated. UVA induced, e.g., OSR-1/STK25, a ROS-activated kinase, and thioredoxin reductase, which together with thioredoxin (Trx) acts at the core of antioxidant defense. β-carotene favored these protective gene regulations.

Overall the data suggest that stress signalling was activated by UVA. β-carotene did not inhibit these UVA effects, and enhanced some.

“UV Response” of Keratinocytes Undergoing β-Carotene-Induced Differentiation Led To Cell Cycle Arrest and Apoptosis

SAPK/JNK signaling often leads to cell cycle arrest and apoptosis. Expression profiles of cell cycle regulators indicated that cell cycle arrest was induced by UVA and further enhanced by β-carotene.

UVA induced several genes which function during the G₁ cell cycle phase (cyclin E, p57^{KIP2}, ornithine decarboxylase). The vast majority of cell cycle regulators functioning in later cell cycle phases were down-regulated by UVA, indicating cell cycle arrest at the late G₁ phase. Examples include the proliferation marker Ki67 and genes involved in DNA replication or encoding mitotic spindle proteins. Moreover, UVA downregulated several growth factor receptors and members of the downstream signalling machinery. β-carotene alone also downregulated genes involved in growth factor signalling, and reduced expression of cell cycle regulators in the context of its differentiation-promoting activity. Combined UVA/β-carotene treatment led to a more pronounced cell cycle arrest than did the single treatments.

Following cell cycle arrest, cells can re-enter the cell cycle or undergo apoptosis. Here, UVA irradiation induced several apoptotic regulators, including the immediate early genes IEX-1, GADD34, GADD153, ERF-2, and TSSC3/IPL. β-carotene enhanced UVA induction of GADD153, GADD34, TDAG51 and ERF-2. The expression profiles of GADD153 and GADD34 were confirmed by QRT-PCR (FIGS. 1b and 1c). The data are consistent with previous evidence that UVA causes apoptosis subsequent to SAPK/JNK activation (see also He, 2004). β-carotene did not reduce this UVA effect. Some gene regulation was enhanced by β-carotene.

Apoptosis induction was confirmed by assessing caspase-3 activity. Caspase-3 activity 5 hours after UVA irradiation was quantified in five separate experiments using the CaspACE™ Assay System (Promega/Catalys, Switzerland). Neither UVA nor β-carotene alone activated caspase-3. β-carotene cooperated with UVA to induce caspase-3 activity in a dose-dependent manner (FIG. 2).

Together, cells pretreated with β-carotene and irradiated with UVA underwent G₁ cell cycle arrest and apoptosis. If this process takes place in vivo β-carotene should favor sun burn cell formation. However, while a mild reduction in sunburn erythema was found in several studies, β-carotene supplementation did not alter the number of sunburn cells in humans (Garmyn, 1995). Induction of apoptosis in the p53-deficient HaCaT cells would imply a favorable removal of precancerous cells, and β-carotene supplementation in most cases indeed reduced skin carcinogenesis in rodents (e.g. Mathews-Roth, 1982). Clinical intervention trials, however, have found no significant prevention of non-melanoma skin cancer (Greenberg, 1990; Green, 1999) by β-carotene. Besides carotenoids, the skin contains other antioxidants, which are believed to prevent β-carotene from enhancing some of the UVA effects in vivo. Furthermore, HaCaT cells are exceptionally sensitive to UV-induced apoptosis (Chaturvedi, 2001). Thus, even though the consequences in skin might be less pronounced than in HaCaT cells, it is possible that the mechanisms identified here nevertheless apply in vivo.

Relationship of the Modes of Action of β-Carotene to its Influence on UVA-Induced Biological Processes

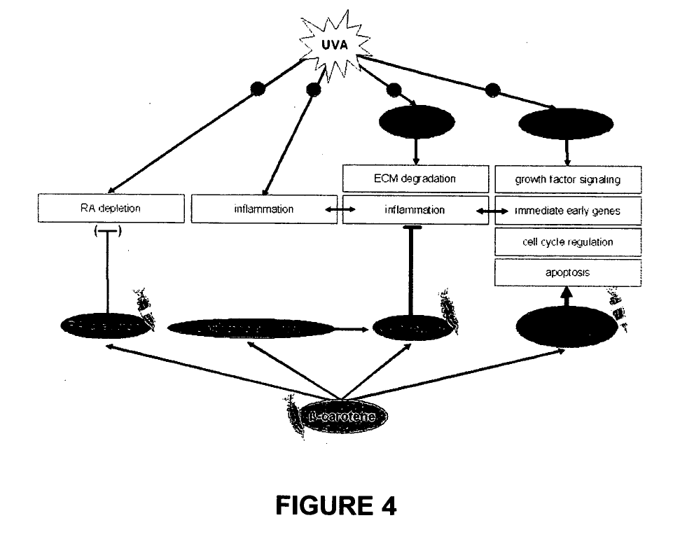

FIG. 4 shows the relationship of the modes of action of β-carotene to its influence on UVA-induced biological processes deduced from the experiments below. β-carotene reduced UVA-induction of genes involved in ECM degradation and inflammation as a ¹O₂ quencher. The mild photoprotective effect of β-carotene appears to be based on inhibition of these ¹O₂-induced gene regulations, rather than on a physical filter effect. A physical filter effect would be expected to reduce all UVA responses by the same amount. β-carotene, if scavenging ROS other than ¹O₂, is irreversibly damaged and converted into radicals, if not rescued by other antioxidants (Edge, 2000). Consistent with this observation, β-carotene did not inhibit UVA-induced stress signals and enhanced some. UVA exposure suppressed several RA target genes. Since HaCaT cells produce marginal amounts of retinoid activity from β-carotene, the provitamin A activity of β-carotene did not translate into restored expression of RA target genes in this system.

β-carotene at physiological concentrations interacted with UVA effects in keratinocytes by multiple mechanisms that included, but were not restricted to ¹O₂ quenching.

In unirradiated keratinocytes, β-carotene reduced expression of immediate early genes, indicating reduced stress signals. Moreover, gene regulation by β-carotene suggested decreased ECM degradation and increased keratinocyte differentiation. This effect on differentiation was unrelated to UVA exposure, but synergized with UVA effects.

In UVA-irradiated cells, β-carotene inhibited gene regulation by UVA, which promoted ECM degradation, indicating a photoprotective effect for β-carotene. β-carotene enhanced UVA-induced PAR-2 expression, suggesting that β-carotene enhanced tanning after UVA exposure. The combination of β-carotene-induced differentiation with the cellular “UV response” led to a synergistic induction of cell cycle arrest and apoptosis by UVA and β-carotene.

The retinoid effect of β-carotene was minor, indicating that the β-carotene effects reported here were predominantly mediated through vitamin A-independent pathways.

The results explain and integrate many conflicting reports on the efficacy of β-carotene as a ¹O₂ quencher and as a general antioxidant in living cells. The mechanisms identified, by which β-carotene acts on the skin, have implications on skin photoaging, as well as on relevant skin diseases, such as skin cancer and psoriasis.

### Example 2

### Quantitative Real Time-Polymerase Chain Reaction

Key gene regulation was confirmed in three independent cell irradiation experiments using TaqMan® QRT-PCR as described (Wertz, 2004). The sequences of the primers and probes used are given in Table 2. In these experiments, cells were pretreated with 0.5, 1.5, or 3 μM β-carotene, to analyze for dose-dependent β-carotene effects. In addition, cells were irradiated either in D₂O-containing PBS or in H₂O-containing PBS, to analyze for the ¹O₂ inducibility of genes.

TABLE 2
Primers and probes used for QRT-PCR.

| | | | |
|---|---|---|---|
| Transcript | Forward Primer | Reverse Primer | Probe |

| | | | |
|---|---|---|---|
| Integrin_{α6} | TTTCCCGTTTCTTTCTTGAGTTGT | TGGAAAAGGTAACTTGTGAGCCA | AGACTCCGTTAGGTTCAGGGAGTTTATCTCCTTTT |
| | (SEQ ID NO: 1) | (SEQ ID NO: 2) | (SEQ ID NO: 3) |
| GADD34 | CGGACCCTGAGACTCCCC | AAGGCCAGAAAGGTGCGCTTCTC | GAAATGGACAGTGACCTTCTCG |
| | (SEQ ID NO: 4) | (SEQ ID NO: 5) | (SEQ ID NO: 6) |
| GADD153 | GCAAGAGGTCCTGTCTTCAGATG | CACCTCCTGGAAATGAAGAGGAAGAATCA | GGGTCAAGAGTGGTGAAGATTTTT |
| | (SEQ ID NO: 7) | (SEQ ID NO: 8) | (SEQ ID NO: 9) |
| 18S rRNA | CGGCTACCACATCCAAGGAA | GCTGGAATTACCGCGGCT | TGCTGGCACCAGACTTGCCCTC |
| | (SEQ ID NO: 10) | (SEQ ID NO: 11) | (SEQ ID NO: 12) |

### BIBLIOGRAPHY

1. Balmer J E, Blomhoff R: Gene expression regulation by retinoic acid. J Lipid Res 43:1773-808, 2002
2. Cantrell A, McGarvey D J, George Truscott T, Rancan F, Bohm F: Singlet oxygen quenching by dietary carotenoids in a model membrane environment. Arch Biochem Biophys 412:47-54, 2003
3. Chaturvedi V, Qin J Z, Denning M F, Choubey D, Diaz M O, Nickoloff B J: Abnormal NF-kappaB signaling pathway with enhanced susceptibility to apoptosis in immortalized keratinocytes. J Dermatol Sci 26:67-78, 2001
4. Edge R, Land E J, McGarvey D J, Burke M, Truscott T G: The reduction potential of the beta-carotene.+/beta-carotene couple in an aqueous micro-heterogeneous environment. FEBS Lett 471:125-7, 2000
5. Fuller C J, Faulkner H, Bendich A, Parker R S, Roe D A: Effect of beta-carotene supplementation on photosuppression of delayed-type hypersensitivity in normal young men. Am J Clin Nutr 56:684-90., 1992
6. Garmyn M, Ribaya-Mercado J D, Russel R M, Bhawan J, Gilchrest B A: Effect of beta-carotene supplementation on the human sunburn reaction. Exp Dermatol 4:104-11., 1995
7. Gille J, Reisinger K, Asbe-Vollkopf A, Hardt-Weinelt K, Kaufmann R: Ultraviolet-A-induced transactivation of the vascular endothelial growth factor gene in HaCaT keratinocytes is conveyed by activator protein-2 transcription factor. J Invest Dermatol 115:30-6, 2000
8. Gollnick. H P M, Hopfenmüller W, Hemmes C, Chun S C, Schmid C, Sundermeier K, Biesalski H K: Systemic β-carotene plus topical UV-sunscreen are an optimal protection against harmful effects of natural UV-sunlight: results of the Berlin-Eilath study. Eur J Dermatol 6:200-205, 1996
9. Green A, Williams G, Neale R, et al.: Daily sunscreen application and betacarotene supplementation in prevention of basal-cell and squamous-cell carcinomas of the skin: a randomised controlled trial. Lancet 354:723-9, 1999
10. Greenberg E R, Baron J A, Stukel T A, et al.: A clinical trial of beta carotene to prevent basal-cell and squamous-cell cancers of the skin. The Skin Cancer Prevention Study Group. N Engl J Med 323:789-95, 1990
11. Grether-Beck S, Olaizola-Horn S, Schmitt H, et al.: Activation of transcription factor AP-2 mediates UVA radiation- and singlet oxygen-induced expression of the human intercellular adhesion molecule 1 gene. Proc Natl Acad Sci USA 93:14586-91, 1996
12. He Y Y, Huang J L, Sik R H, Liu J, Waalkes M P, Chignell C F: Expression profiling of human keratinocyte response to ultraviolet A: implications in apoptosis. J Invest Dermatol 122:533-43, 2004
13. Hughes D A: Dietary carotenoids and human immune function. Nutrition 17:823-7, 2001
14. Kick G, Messer G, Plewig G, Kind P, Goetz A E: Strong and prolonged induction of c-jun and c-fos proto-oncogenes by photodynamic therapy. Br J Cancer 74:30-6, 1996
15. Krinsky N I, Yeum K J: Carotenoid-radical interactions. Biochem Biophys Res Commun 305:754-60, 2003
16. Mathews-Roth M M: Antitumor activity of beta-carotene, canthaxanthin and phytoene. Oncology 39:33-7, 1982
17. Mathews-Roth M M, Pathak M A, Parrish J, Fitzpatrick T B, Kass E H, Toda K, Clemens W: A clinical trial of the effects of oral beta-carotene on the responses of human skin to solar radiation. J Invest Dermatol 59:349-53, 1972
18. Postaire E, Jungmann H, Bejot M, Heinrich U, Tronnier H: Evidence for antioxidant nutrients-induced pigmentation in skin: results of a clinical trial. Biochem Mol Biol Int 42:1023-33, 1997
19. Raap W P, Tronnier H, Wiskemann A: Photoprotection and skin coloring by oral carotenoids. Dermatologica 171:371-3, 1985
20. Siler U, Barella L, Spitzer V, Schnorr J, Lein M, Goralczyk R, Wertz K: Lycopene and vitamin E interfere with autocrine/paracrine loops in the Dunning prostate cancer model. Faseb J 18:1019-21, 2004
21. Sorg O, Tran C, Carraux P, Didierjean L, Falson F, Saurat J H: Oxidative stress-independent depletion of epidermal vitamin A by UVA. J Invest Dermatol 118:513-8, 2002
22. Stahl W, Heinrich U, Jungmann H, Sies H, Tronnier H: Carotenoids and carotenoids plus vitamin E protect against ultraviolet light-induced erythema in humans. Am J Clin Nutr 71:795-8, 2000
23. Thurmann P A, Steffen J, Zwernemann C, Aebischer C P, Cohn W, Wendt G, Schalch W: Plasma concentration response to drinks containing beta-carotene as carrot juice or formulated as a water dispersible powder. Eur J Nutr 41:228-35, 2002
24. Tronnier H: Protective effect of beta-carotene and canthaxanthin against UV reactions of the skin. Z Hautkr 59:859-70, 1984
25. Wertz K, Seifert N, Buchwald Hunziker P, Riss G, Wyss A, Lankin C, Goralczyk R: β-carotene inhibits UVA-induced MMP 1 and expression in keratinocytes by a singlet oxygen-dependent mechanism. Free Radic Biol Med 37:654-670, 2004
26. Wlaschek M, Heinen G, Poswig A, Schwarz A, Krieg T, Scharffetter-Kochanek K: UVA-induced autocrine stimulation of fibroblast-derived collagenase/MMP-1 by interrelated loops of interleukin-1 and interleukin-6. Photochem Photobiol 59:550-6, 1994

The scope of the present invention is not limited by the description, examples, and suggested uses herein and modifications can be made without departing from the spirit of the invention. Thus, it is intended that the present invention cover modifications and variations of this invention provided that they come within the scope of the appended claims and their equivalents.

1. A method for screening for a compound that modulates an effect of UV irradiation on eukaryotic cells comprising:
a) contacting a sample of eukaryotic cells with a compound to be evaluated;
b) irradiating the cells from a) with UV radiation;
c) comparing a gene expression profile of the cells contacted with the compound to a gene expression profile of control cells that were not contacted with the compound prior to the irradiation step in b);
d) correlating a difference in the gene expression profile of the cells exposed to the compound and the control cells that were not exposed to the compound with an ability of the compound to modulate an effect of UV irradiation on the cells.

2. A method according to claim 1, wherein the gene expression profile comprises an expression profile of a gene selected from the group consisting of immediate early genes, oxidative defense genes, extracellular matrix genes, pro-inflammatory genes, VEGF-related ligand and receptor genes, IFNα/β genes, interleukin genes, proteinase-activated receptor genes, prostaglandin synthesis and signalling genes, EGF-related ligand and receptor genes, FGF-related ligand and receptor genes, TGF-β-related ligand and receptor genes, Wnt signalling genes, IGF/insulin signalling genes, Jagged/Delta signalling genes, MAPK pathway genes, differentiation marker genes, cell cycle genes, apoptosis genes, and combinations thereof.

3. A method according to claim 2, wherein the gene expression profile comprises an expression profile of a gene selected from the group consisting of C-FOS, FRA-1, JUN-D, JUN-B, MAF-F, C-MYC, OSR-1, GEM, DKK-1, GADD34, GADD153, IEX-1, TSSC3/IPL, TDAG51, MMP-1, MMP-3, MMP-10, serpinB1, lekti, PAR-2, VEGF, IL-6, HB-EGF, SMADs, EGFR HER3, Wnt5A, FGFR2, cyclin E, ODC, ID1-3, ID-4, RB, K167, thymidylate synthase, DNA ligase III, CENP-E, centormere and spindle protein genes, COL4, COL7, Cx31, BPAG1, integrin α6, KLF4, ILK, and combinations thereof.

4. A method according to claim 1, wherein the gene expression profile is determined by screening an array or a microarray.

5. A method according to claim 1, wherein the gene expression profile is determined by real time polymerase chain reaction.

6. A method according to claim 1, wherein the UV radiation is UV A radiation.

7. A method according to claim 1, wherein the eukaryotic cells are keratinocytes.

8. A method according to claim 7, wherein the keratinocytes are obtained from a culture of HaCaT cells.

9. A method for ameliorating the effects of non-UV radiation induced skin aging comprising administering to an organism in need thereof an amount of a compound selected from the group consisting of β-carotene, a precursor of β-carotene, a derivative of β-carotene, a salt of β-carotene, and combinations thereof, which amount is effective to modulate a gene responsible for the non-UV radiation induced skin aging.

10. A method according to claim 9, wherein the gene responsible for non-UV radiation skin aging is selected from the group consisting of a member of the stress signal family of genes, a member of the ECM degradation family of genes, a member of the immune modulation family of genes, a member of the inflammation-causing family of genes, a member of the cellular differentiation family of genes, and combinations thereof

11. A method according to claim 10, wherein the cellular differentiation family of genes is selected from the group consisting of growth factor signalling genes, cell cycle regulation genes, differentiation genes, apoptosis genes, and combinations thereof.

12. A method according to claim 11, wherein the growth factor signalling genes are selected from the group consisting of EGFR, HER-3, FGF3, FRZ-6, NOTCH3, BMP2a, Wnt5a, and combinations thereof and the cell cycle regulation genes are selected from the group consisting of G1, RB, p21, ID-2, DNA ligase III, DNA-PK G2/M, BUB1, and combinations thereof.

13. A method according to claim 10, wherein the immune modulation and inflammation family of genes are selected from the group consisting of VEGF, IL-18, COX-2, and combinations thereof.

14. A method according to claim 10, wherein the ECM degradation family of genes is selected from the group consisting of MMP-1, MMP-10, and combinations thereof.

15. A method according to claim 10, wherein the stress signal family of genes is selected from the group consisting of JUN-B, FRA-2, NRF-2, GEM, EGRα, TSSC3/IPL, and combinations thereof.

16. A method according to claim 9, wherein the organism is a human.

17. A method according to claim 9, wherein the compound is administered in an amount from about 1 to about 30 mg per day.

18. A composition for ameliorating the effects of non-UV radiation induced skin aging comprising an amount of a compound selected from the group consisting of β-carotene, a precursor of β-carotene, a derivative of β-carotene, a salt of β-carotene, and combinations thereof, which amount is effective to modulate a gene responsible for the non-UV radiation induced skin aging.

19. A composition according to claim 18, wherein the gene responsible for non-UV radiation skin aging is selected from the group consisting of a member of the stress signal family of genes, a member of the ECM degradation family of genes, a member of the immune modulation family of genes, a member of the inflammation-causing family of genes, a member of the cellular differentiation family of genes, and combinations thereof

20. A composition according to claim 19, wherein the cellular differentiation family of genes is selected from the group consisting of growth factor signalling genes, cell cycle regulation genes, differentiation genes, apoptosis genes, and combinations thereof.

21. A composition according to claim 20, wherein the growth factor signalling genes are selected from the group consisting of EGFR, HER-3, FGF3, FRZ-6, NOTCH3, BMP2a, Wnt5a, and combinations thereof and the cell cycle regulation genes are selected from the group consisting of G1, RB, p21, ID-2, DNA ligase III, DNA-PK G2/M, BUB1, and combinations thereof.

22. A composition according to claim 19, wherein the immune modulation and inflammation family of genes are selected from the group consisting of VEGF, IL-18, COX-2, and combinations thereof.

23. A composition according to claim 19, wherein the ECM degradation family of genes is selected from the group consisting of MMP-1, MMP-10, and combinations thereof.

24. A composition according to claim 19, wherein the stress signal family of genes is selected from the group consisting of JUN-B, FRA-2, NRF-2, GEM, EGRα, TSSC3/IPL, and combinations thereof.

25. A composition according to claim 18, wherein the amount of the compound in the composition is from about 1 to about 30 mg.

26. A composition according to claim 18, which is in a dosage form selected from the group consisting of a dietary supplement, a food, a feed, a beverage, a fortified food, a fortified feed, a pharmaceutical, a personal care product, a nutraceutical, a functional food, a functional feed, a clinical nutrition product, a food additive, and a feed additive.

27. A method of modulating the effects of UVA-induced gene expression on skin aging comprising: prior to exposure to UVA radiation, administering to an organism an amount of a composition comprising a compound selected from the group consisting of β-carotene, a precursor of β-carotene, a derivative of β-carotene, a salt of β-carotene, and combinations thereof, which amount is effective to modulate the effects of UVA-induced gene expression on skin aging.

28. A method according to claim 27, wherein the organism is a human.

29. A method according to claim 27, wherein the amount effective to modulate the effects of UVA-induced gene expression on skin aging is sufficient to:
(a) quench UV-A induced expression of a gene selected from the group consisting of MMP-10, VEGF, IFNα/B targets, ID-4, and combinations thereof;
(b) down regulate UV-A induced expression of a gene selected from the group consisting of JUN-B, EGFR, HER3, FGFR2, SMADs, WNT-5a, IDI-3, RB8, thymidylate synthase, DNA ligase G2/M, CENP-E, centromere proteins, spindle proteins, and combinations thereof; or
(c) upregulate UV-A induced expression C-FOS, FRA-1, JUN-D, MAF-F, C-MYC, OSR-1, GEM, DKK-1, GADD34, GADD153, IEX-1, TSSC3/MPL, TDAG51, serpinB1, lekti, PAR-2, IL-6, HB-EGF, G1, cyclinE, ODC, Cx31, KLF4, GADD153, and combinations thereof.

30. A method according to claim 27, wherein the compound is present in the composition in an amount from about 1 to about 30 mg per day.

31. A composition for modulating the effects of UVA-induced gene expression on skin aging comprising: an amount of a compound selected from the group consisting of β-carotene, a precursor of β-carotene, a derivative of β-carotene, a salt of β-carotene, and combinations thereof, which amount is effective to modulate the effects of UVA-induced gene expression on skin aging.

32. A composition according to claim 31, wherein the amount of the compound is effective to:
(a) quench UV-A induced expression of a gene selected from the group consisting of MMP-10, VEGF, IFNα/B targets, ID-4, and combinations thereof;
(b) down regulate UV-A induced expression of a gene selected from the group consisting of JUN-B, EGFR, HER3, FGFR2, SMADs, WNT-5a, IDI-3, RB8, thymidylate synthase, DNA ligase G2/M, CENP-E, centromere proteins, spindle proteins, and combinations thereof; or
(c) upregulate UV-A induced expression C-FOS, FRA-1, JUN-D, MAF-F, C-MYC, OSR-1, GEM, DKK-1, GADD34, GADD153, IEX-1, TSSC3/MPL, TDAG51, serpinB1, lekti, PAR-2, IL-6, HB-EGF, G1, cyclinE, ODC, Cx31, KLF4, GADD153, and combinations thereof.

33. A composition according to claim 31, wherein the amount of the compound present in the composition is from about 1 to about 30 mg.

34. A composition according to claim 31, which is in a dosage form selected from the group consisting of a dietary supplement, a food, a feed, a beverage, a fortified food, a fortified feed, a pharmaceutical, a personal care product, a nutraceutical, a functional food, a functional feed, a clinical nutrition product, a food additive, and a feed additive.

35. A method of enhancing UVA-induced tanning of the skin comprising: administering to an organism, prior to exposure to UVA radiation, an amount of a composition comprising a compound selected from the group consisting of β-carotene, a precursor of β-carotene, a derivative of β-carotene, a salt of β-carotene, and combinations thereof, which amount is effective to increase UVA-induced PAR-2 gene transcription.

36. A method according to claim 35, wherein the organism is a human.

37. A method according to claim 35, wherein the compound is present in the composition in an amount from about 1 to about 30 mg per day.

38. A composition for enhancing UVA-induced tanning comprising an amount of a compound selected from the group consisting of β-carotene, a precursor of β-carotene, a derivative of β-carotene, a salt of 8-carotene, and combinations thereof, which amount is effective to increase UVA-induced PAR-2 gene transcription.

39. A composition according to claim 38, wherein the amount of the compound present in the composition is from about 1 to about 30 mg.

40. A composition according to claim 38, which is in a dosage form selected from the group consisting of a dietary supplement, a food, a feed, a beverage, a fortified food, a fortified feed, a pharmaceutical, a personal care product, a nutraceutical, a functional food, a functional feed, a clinical nutrition product, a food additive, and a feed additive.

41. A method for promoting cell differentiation in UVA-irradiated cells of an organism comprising administering to the organism in need thereof an amount of a compound selected from the group consisting of β-carotene, a precursor of β-carotene, a derivative of β-carotene, a salt of β-carotene, and combinations thereof, which amount is effective to downregulate transcription of a gene selected from the group consisting of BPAG1, integrin_{α6}, ILK, desmocollins, Cx45 and combinations thereof or to up regulate transcription of a gene selected from the group consisting of Cx31, KLF4, GADD153, and combinations thereof.

42. A method according to claim 41, wherein the organism is a human.

43. A method according to claim 41, wherein the compound is administered in an amount from about 1 to about 30 mg per day.

44. A composition for promoting cell differentiation in UVA irradiated cells of an organism comprising an amount of a compound selected from the group consisting of β-carotene, a precursor of β-carotene, a derivative of β-carotene, a salt of β-carotene, and combinations thereof, which compound is effective to downregulate transcription of a gene selected from the group consisting of BPAG1, integrin_{α6}, ILK, desmocollins, Cx45, and combinations thereof or upregulate transcription of a gene selected from the group consisting of Cx31, KLF4, GADD153, and combinations thereof.

45. A composition according to claim 44, wherein the amount of the compound present in the composition is from about 1 to about 30 mg.

46. A composition according to claim 44, which is in a dosage form selected from the group consisting of a dietary supplement, a food, a feed, a beverage, a fortified food, a fortified feed, a pharmaceutical, a personal care product, a nutraceutical, a functional food, a functional feed, a clinical nutrition product, a food additive, and a feed additive.

47. A method for modulating stress-induced induction of a gene in an organism comprising: administering to the organism an amount of a compound selected from the group consisting of β-carotene, a precursor of β-carotene, a derivative of β-carotene, a salt of β-carotene, and combinations thereof, which amount is effective to modulate the stress-induced induction of the gene.

48. A method according to claim 47, wherein the gene is selected from the group consisting of an immediate early gene, an oxidative stress defense gene, and combinations thereof.

49. A method according to claim 48, wherein the immediate early gene is selected from the group consisting of GEM, KRS-2, JUN-B, FRA-2, EGRα, and combinations thereof and the oxidative stress defense gene is selected from the group consisting of NCF2, NRF2β1, and combinations thereof.

50. A method according to claim 47, wherein the organism is a human.

51. A method according to claim 47, wherein the compound is administered to the organism in an amount from about 1 to about 30 mg per day.

52. A method according to claim 47, wherein the modulation comprises down regulation of one or more genes selected from the group consisting of the immediate early gene family, the oxidative stress defense gene family, and combinations thereof.

53. A method according to claim 47, wherein the stress stimuli comprises oxidative stress.

54. A method according to claim 47, wherein the stress stimuli comprises UV irradiation.

55. A composition for modulating stress-induced induction of a gene in an organism comprising a compound selected from the group consisting of β-carotene, a precursor of β-carotene, a derivative of β-carotene, a salt of β-carotene, and combinations thereof, wherein the compound is present in the composition in an amount effective to modulate the stress-induced induction of the gene.

56. A composition according to claim 55, wherein the gene is selected from the group consisting of an immediate early gene, an oxidative stress defense gene, and combinations thereof.

57. A composition according to claim 56, wherein the immediate early gene is selected from the group consisting of GEM, KRS-2, JUN-B, FRA-2, EGRα, and combinations thereof and the oxidative stress defense gene is selected from the group consisting of NCF2, NRF2β1, and combinations thereof.

58. A composition according to claim 55, wherein the amount of the compound in the composition is from about 1 to about 30 mg.

59. A composition according to claim 55, which is in a dosage form selected from the group consisting of a dietary supplement, a food, a feed, a beverage, a fortified food, a fortified feed, a pharmaceutical, a personal care product, a nutraceutical, a functional food, a functional feed, a clinical nutrition product, a food additive, and a feed additive.


SEQUENCE LISTING (12 sequences)

SEQ ID NO: 1 - DNA, 24 bp, Artificial
  Forward primer for Integrin-alpha6 RNA in the human keratinocyte cell line HaCaT.
gggtcaagagtggtgaagattttt

SEQ ID NO: 2 - DNA, 23 bp, Artificial
  Reverse primer for Integrin-alpha6 RNA in the human keratinocyte cell line HaCaT.
tggaaaaggtaacttgtgagcca

SEQ ID NO: 3 - DNA, 35 bp, Artificial
  Probe for Integrin-alpha6 RNA in the human keratinocyte cell line HaCaT.
agactccgttaggttcagggagtttatctcctttt

SEQ ID NO: 4 - DNA, 18 bp, Artificial
  Forward primer for GADD34 RNA in the human keratinocyte cell line HaCaT.
cggaccctgagactcccc

SEQ ID NO: 5 - DNA, 23 bp, Artificial
  Reverse primer for GADD34 RNA in the human keratinocyte cell line HaCaT.
aaggccagaaaggtgcgcttctc

SEQ ID NO: 6 - DNA, 22 bp, Artificial
  Probe for GADD34 RNA in the human keratinocyte cell line HaCaT.
gaaatggacagtgaccttctcg

SEQ ID NO: 7 - DNA, 23 bp, Artificial
  Forward primer for gadd153 RNA in the human keratinocyte cell line HaCaT.
gcaagaggtcctgtcttcagatg

SEQ ID NO: 8 - DNA, 29 bp, Artificial
  Reverse primer for GADD153 RNA in the human keratinocyte cell line HaCaT.
cacctcctggaaatgaagaggaagaatca

SEQ ID NO: 9 - DNA, 24 bp, Artificial
  Probe for GADD153 RNA in the human keratinocyte cell line HaCaT.
gggtcaagagtggtgaagattttt

SEQ ID NO: 10 - DNA, 20 bp, Artificial
  Forward primer for 18S rRNA in the human keratinocyte cell line HaCaT.
cggctaccacatccaaggaa

SEQ ID NO: 11 - DNA, 18 bp, Artificial
  Reverse primer for 18S rRNA in the human keratinocyte cell line HaCaT.
gctggaattaccgcggct

SEQ ID NO: 12 - DNA, 22 bp, Artificial
  Probe for 18S rRNA in the human keratinocyte cell line HaCaT.
tgctggcaccagacttgccctc